(19) United States
(12) Patent Application Publication
(10) Pub. No.: US 2004/0202701 A1
(43) Pub. Date: Oct. 14, 2004

(54) Modified siloxane yielding transferring benefits from soft tissue products

(75) Inventors: Dale A. Burghardt, Butte des Morts, WI (US); Duane G. Krzysik, Appleton, WI (US); David R. Otts, Appleton, WI (US); Cynthia W. Henderson, Neenah, WI (US); Bernard J. Minerath III, Oshkosh, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/757,834

(22) Filed: Jan. 15, 2004

Related U.S. Application Data
(63) Continuation of application No. 09/742,666, filed on Dec. 21, 2000, now abandoned.
(60) Provisional application No. 60/173,195, filed on Dec. 27, 1999.

(51) Int. Cl. A61K 7/06; A61K 7/11; A61K 9/70

(52) U.S. Cl. 424/443; 424/70.12

(57) ABSTRACT

A paper product, such as a facial tissue, that is capable of transferring beneficial chemical agents to the user is disclosed. In one embodiment, the paper product is treated with a siloxane treatment that contains two (2) siloxanes. The first siloxane bonds to pulp fibers contained within the paper product. The second siloxane composition, on the other hand, is bonded to a beneficial chemistry agent. When contacted with a user's skin, the second siloxane composition bonded to the beneficial chemistry agent is transferred to the user's skin.

### RELATED APPLICATIONS

The present application is based on a Provisional Application having Serial No. 60/173,195, which was filed on Dec. 27, 1999. The present application is also a Continuation Application of U.S. patent application Ser. No. 09/742,666 now abandoned.

### FIELD OF THE INVENTION

The present invention is generally directed to a facial tissue and a method for producing a facial tissue which is capable of transferring beneficial chemistries to a consumer's skin. More specifically, the present invention is directed to a method for producing a facial tissue where a modified siloxane chain attached to some beneficial chemistry or chemistries is used to treat the surface of a tissue or other nonwoven material.

### BACKGROUND OF THE INVENTION

Consumers use paper wiping products, such as facial tissues, for a wide variety of applications. For example facial tissues are not only used for nose care, but are also used as a general wiping product, as a product used in conjunction with cosmetics, and for eyeglass cleaning etc. Consequently, there are many different types of tissue products currently commercially available.

In one embodiment, tissue products are treated with siloxanes that increase the softness of the facial tissue. Adding such a silicone compound to a facial tissue imparts improved softness to the tissue while maintaining the tissue's absorbency and strength and while reducing the amount of lint produced by the tissue during use. Recent developments in adding a silicone compound to a facial tissue in order for the tissue to exhibit improved softness and reduced lint while maintaining absorbency are disclosed in U.S. Pat. No. 5,227,242 entitled “Multifunctional Facial Tissue” and U.S. Pat. No. 4,950,545 both assigned to the assignee of the present invention and which are both incorporated by reference.

Although the above identified patents provide great improvements in the art, various deficiencies still remain. For instance, the prior art is generally deficient in providing paper wiping products, such as facial tissues, that are capable of transferring beneficial chemistries, such as anti-inflammatory agents, lipids, protease inhibitors, sequestration agents and the like to the skin of a user. In this regard, a need currently exists for a paper wiping product having good softness, absorbency and strength characteristics that is also capable of transferring beneficial chemistries to a user's skin.

### SUMMARY OF THE INVENTION

The present invention recognizes and addresses various drawbacks and deficiencies of prior art constructions and methods.

Accordingly, it is an object of the present invention to provide an improved wiping product, such as a facial tissue.

Another object of the present invention is to provide an improved paper product, such as a facial tissue, that is capable of transferring beneficial compounds to an opposing surface, such as a user's skin.

These and other objects of the present invention are achieved by providing a paper product capable of transferring beneficial chemical compounds to an opposing surface. The paper product includes a nonwoven web containing pulp fibers. A siloxane treatment is applied to at least one surface of the nonwoven web. The siloxane treatment includes an amino functional siloxane composition which will bond to the pulp fibers contained in the web.

Further, the siloxane treatment includes a complex of a non-amino functional siloxane and a beneficial chemistry agent. The non-amino functional siloxane and beneficial chemistry agent complex has a charge attraction to the amino functional siloxane composition. Of particular advantage, when an opposing surface is contacted with the paper product, the complex is configured to be transferred to the opposing surface.

The amino functional siloxane composition used in the present invention can an amine-modified polysiloxane. The amine-modified polysiloxane can have viscosity of from about 25 centipoise to about 200,000 centipoise.

The non-amino functional siloxane that may be used in the present invention, on the other hand, can be an alkyl siloxane, a hydroxyl siloxane, or hydrogen saturated siloxane. Particular examples include methyl dimethyl siloxane and a polydimethyl siloxane.

As used herein, a beneficial chemistry agent refers to any agent that can have a medicinal value, therapeutic value or can have any type of antimicrobial effect. Examples of beneficial chemistry agents that may be used in the present invention include anti-inflammatory compounds, lipids, inorganic anions, inorganic cations, protease inhibitors, sequestration agents and mixtures thereof.

Preferably, the siloxane treatment of the present invention is contained in an emulsion and applied to the surface of the nonwoven web. For instance, the siloxane treatment can be printed onto the web. The siloxane treatment can be applied to the web in an amount from about 0.1 percent to about 50 percent by weight, an particularly from about 0.1 percent to about 5 percent by weight.

Although any suitable paper product may be made in accordance with the present invention, facial tissues are particularly well suited for receiving the siloxane treatment. Facial tissue, for instance, can be a single ply or a multi ply tissue.

In an alternative embodiment of the present invention, the siloxane treatment containing the beneficial chemistry agent can be combined with a lotion composition and applied to a surface of a nonwoven web. In this embodiment, the siloxane treatment need only contain the non-amino functional siloxane and beneficial chemistry agent complex. When the nonwoven web is contacted against an opposing surface, the complex transfers to the opposing surface along a portion with lotion composition.

The lotion composition combined with the siloxane treatment can vary depending the particular application. For instance, the lotion composition can be water based or oil based. The lotion composition can be applied to the nonwoven web in an amount from about 0.5 percent to about 40 percent by weight, and particularly from about 3 percent to about 15 percent.

In one embodiment, the lotion composition comprises an oil and a wax. The oil can be present in the lotion composition in an amount from about 30 percent to about 90 percent by weight, and particularly from 40 percent to about 70 percent by weight. The wax, on the other hand, can be contained within the lotion composition in an amount from about 10 percent to about 40 percent by weight, and particularly from about 10 percent to about 30 percent by weight.

Besides an oil and a wax, the lotion composition can also contain a fatty alcohol. The fatty alcohol can be contained within the composition in an amount from about 5 percent to about 40 percent by weight. Preferably, the fatty alcohol contains a carbon chain length of from about 14 carbon atoms to about 30 carbo atoms.

Other objects, features and aspects of the present invention are discussed in greater detail below.

### DETAILED DESCRIPTION OF PREFERRED EMBODIMENTS

It is to be understood by one of ordinary skill in the art that the present discussion is a description of exemplary embodiments only, and is not intended as limiting the broader aspects of the present invention, which broader aspects are embodied in the exemplary construction.

In general, the present invention is directed to various paper products, such as facial tissues, having great softness characteristics and having the ability to transfer beneficial chemistries to a consumer.

More particularly, the present invention is directed to modifying a siloxane chain with chemical compounds that are beneficial to the consumer and then applying the modified siloxane to the surface of a facial tissue or other nonwoven material. The beneficial chemistry agent can then be transferred from the tissue to the consumer in efficacious or functional amounts during use. In general, any type of beneficial chemical compound may be used in the present invention, including anti-inflammatory agents, lipids, inorganic anions and cations, protease inhibitors, sequestration agents, and the like.

The above-described siloxane treatment can be applied to a facial tissue, bath tissue, kitchen towels, any other tissue product, or possibly any other nonwoven material. It is believed that the modified siloxane will attach to virtually any type of material.

In one embodiment, the paper product, such as a tissue product, is treated with at least two siloxane compounds. The first siloxane compound comprises a siloxane that will bond to cellulosic fibers, such as pulp fibers. The second siloxane compound, on the other hand, is bonded to one or more beneficial chemistry agents to form a complex. As used herein, a complex refers to a chemical association formed between two compounds which can be, for instance, a covalent bond, an ionic bond or the like. Of particular advantage, it has been discovered that siloxanes will bond and form a complex with virtually any type of beneficial chemistry agent due to the ease in which silicone bonds with other elements.

According to the present invention, the siloxanes are combined to form an emulsion and applied to a paper product or, alternatively, are added to the paper product in separate emulsions. When applied to the paper product, the first siloxane bonds to cellulosic fibers contained within the paper product which increases the softness and improves the surface feel of the base sheet. The second siloxane bound to one or more beneficial chemistry agents, on the other hand, resides on the surface of the paper product and forms a charge attraction with the first siloxane. In accordance with the present invention, it has been discovered that when the paper product is then applied to an adjacent surface, such as to a user's skin, the second siloxane-beneficial chemistry agent complex transfers to the opposing surface due to the force friction.

In one embodiment, the first siloxane composition can be a generally hydrophobic amino functional siloxane or similar silicone that is capable of bonding to the surface of the baseweb. Such an amino functional siloxane may have the following general formula:

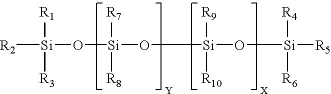

wherein x and y are integers >0 and the mole ratio of x to (x+y) is from about 0.005 percent to about 25 percent. The R₁-R₉ moieties can be C₁ or greater alkyl substituents. Additionally, R₂ and R₅ can be hydroxyl or C, or greater alkyl alcohol substituents. Preferred R₁-R₉ moieties include C₁-C₄. The R₁₀ moiety can include any amine-related functional group or groups such as an amine, imine, and/or amide.

For example, the amine-modified polysiloxane can be a polysiloxane where the R₁₀ moiety contains one amine group per substituent or two or more amine groups per substituent, separated by a linear or branched alkyl chain of C₁ or greater.

Modified polysiloxane materials which are suitable for blending or mixing with the amine-modified polysiloxane(s) for purposes of balancing the hydrophobicity in accordance with this invention have the following general formula:

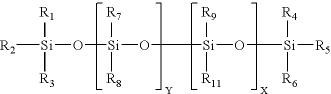

wherein x and y are integers >0. The mole ratio of x to (x+y) can be from 0.005 percent to about 25 percent. The R₁-R₉ moieties can be C₁ or greater alkyl substituents. Additionally, R₂ and R₅ can be hydroxyl or C₁ or greater alkyl alcohol substituents. Preferred R₁-R₉ moieties include C₁-C₄. The R₁₁ moiety can include organic functional groups such as ether, polyether, ester, amine, imine, amide, or other functional groups, including the alkyl and alkenyl analogues of such functional groups.

As an example, the R₁₁ moiety can be a polyether functional group of the generic form —R₁₂—(R₁₃—O)ₐ—(R₁₄—O)_{b}—R₁₅; wherein R₁₂, R₁₃ and R₁₄ are alkyl chains of C₁ or greater, R₁₅ can be Hydrogen or a C₁-C₄ alkyl group, and “a” and “b” can be integers of from 1-100, more specifically from 10-30.

The viscosity range of the amine-modified polysiloxane, which is indicative of the molecular weight, can be from about 25 centipoise to about 2,000,000 centipoise or higher, more specifically from about 100 to about 1,000,000 centipoise.

In accordance with the present invention, once a first siloxane composition has been selected that is capable of bonding to pulp fibers, a second siloxane composition is chosen that is capable of bonding to beneficial chemistry agents and that can be combined with the first siloxane. In one embodiment, when the first siloxane composition is an amino functional siloxane, the second siloxane composition can be a generally hydrophilic non-amino functional siloxane, which has been modified to incorporate one or more beneficial chemical compounds. Non-amino functional siloxanes do not have a high affinity for bonding with the paper fiber of the tissue product but are attracted to the first siloxane composition.

Several examples of non-amino functional siloxanes that can be modified by a chemically beneficial additive include methyl dimethyl siloxane, polydimethyl siloxane, other alkyl siloxanes, siloxanes attached to hydroxyl groups, and siloxanes attached to hydrogen.

The combination of an amino functional siloxane and a modified non-amino functional siloxane forms a siloxane emulsion that may be applied to a facial tissue or other nonwoven material at one time. Normally, the combination of siloxanes (one amino functional and one non-amino functional) is emulsified in water using an appropriate surfactant before the combination is applied to the surface of the tissue. However, in an alternative embodiment, the siloxanes are applied to the facial tissue at separate times in that the amino functional siloxane is first applied to the surface and then the non-amino functional siloxane (modified by the beneficial chemical compound) is applied.

Either the emulsion of both the siloxanes or each siloxane separately may be applied to a facial tissue or other nonwoven by printing, spraying, dipping, coating or the like. For most applications, the siloxane emulsion or single siloxanes are incorporated into the facial tissue or other nonwoven material after the product has been formed.

In a preferred embodiment of the present invention, either the siloxane emulsion or the single siloxanes in the sequential order described above are printed onto a dried, creped tissue sheet between the base sheet manufacturing process and the final tissue product converting process. Printing provides precise control of the add-on amount of the siloxane mixture and places the siloxane mixture on the surface of the tissue where it is most effective for transferability to the consumer's skin. Also, printing provides a distinct pattern of treated areas and untreated areas, thereby mitigating any decrease in absorbency attributable to the presence of siloxane compounds. More specifically, gravure printing is preferred because of the control it offers with respect to the amount of siloxane added to the tissue surface.

The base sheet that is treated with the siloxane composition preferably contains pulp fibers. As described above, the base sheet can be a paper towel, a bath tissue, a facial tissue, any other tissue product, or possible any nonwoven material. For most applications, however, a facial tissue will be treated in accordance with the present invention. The facial tissue can have a density of from about 0.04 grams per cubic centimeter to about 0.3 grams per cubic centimeter and can have a basis weight of from about 4 to about 40 pounds per ream (2,880 square feet). Tensile strength in the machine direction can vary but will generally be in the range of from about 100 to about 5,000 grams per inch of width. Tensile strength in the cross-machine direction can be in the range of from about 50 grams to about 2,500 grams per inch of width. Absorbency for tissue products is typically at least about 5 grams of water per gram of fiber, and generally from about 5 to about 9 grams of water per gram of fiber.

The tissue sheets can be made from various materials including natural cellulosic fiber sources such as hard woods, soft woods and nonwoody species, but can also contain significant amounts of recycled fibers, sized or chemically-modified fibers, or synthetic fibers. The tissue product can be a single ply product or can be a multi-ply product.

In one embodiment, the siloxane treatment of the present invention is printed on to a multi-ply facial tissue. This is accomplished by first unwinding two rolls of single-ply facial grade creped tissue and then crimping the two together at a given speed. The single-ply facial grade creped tissue may be a blend of softwood fibers and hardwood fibers. The resulting two-ply base sheet is then treated with the siloxane emulsion (or with each individual siloxane) on both sides using a rotogravure printer, first printing on one side and then the other. Such a rotogravure printer produces a printing pattern that is uniform and provided by printing cells of a certain micron size and spaced apart by a certain number of microns.

Alternative embodiments of the present invention, wherein either the siloxane emulsion or each individual siloxane is applied to a facial tissue or other nonwoven material by spraying, dipping, or coating serve to cover the entire surface of the facial tissue or other nonwoven material with the modified siloxane emulsion containing beneficial chemical compounds. This differs from the abovedescribed embodiment of the present invention in that the printing application of the siloxane treatment produces a pattern of treated and untreated areas rather than covering the entire surface.

The amount of the siloxane treatment that is applied to a paper product in accordance with the present invention will generally vary with depending upon the product being treated, the composition of the siloxane treatment, the beneficial chemical agents used, the particular application, and desired result. For most applications, however, the siloxane treatment is added to a base web in an amount from about 0.1 percent by weight to about 50 percent by weight, and particularly from about 0.1 percent by weight to about 5.0 percent by weight based on the dry weight of the paper product.

The amino functional siloxane or similar silicone bonds to the surface of the tissue product while the modified non-amino functional siloxane resides on the surface of the tissue product near the other siloxane due to the attraction of silicones. The non-amino functional siloxane is then transferable by friction to the consumer, and the beneficial chemical compound attached to the non-amino functional siloxane contacts the consumer's skin and imparts certain benefits to the consumer.

The beneficial chemical agents that can be used in the present invention include skin wellness chemistries or health agents including but not limited to anti-inflammatory compounds, lipids, inorganic anions and cations, protease inhibitors, or sequestration agents. Furthermore, as additional beneficial chemical compounds are identified, such compounds may be attached to a siloxane chain, applied to a facial tissue, and then transferred to the consumer's skin.

An example of an anti-inflammatory additive that might be bonded to the non-amino functional siloxane is cortisone. Lipid additives, which can be transferred from a facial tissue to the consumer, serve as good healing agents.

Examples of inorganic cations that may be used as additives to be transferred from the facial tissue to the consumer include copper ions and zinc ions. The protease inhibitors combat irritants such as those irritants found in nasal discharge. Further, sequestration agents bonded to the non-amino functional siloxane are useful in that they fasten onto harmful compounds (for example, metal chelates) so that such harmful compounds are no longer on the consumer's skin.

Other ingredients and their corresponding benefits include, without limitation, anti-acne actives (a drug product used to reduce the number of acne blemishes, acne pimples, blackheads, and whiteheads), antimicrobial actives, antifungal actives, antiseptic actives, antioxidants, cosmetic astringents (include a tightening or tingling sensation on skin), drug astringents (a drug product which checks oozing, discharge, or bleeding when applied to skin or mucous membrane and works by coagulating protein), biological additives (enhance the performance or consumer appeal of the product), deodorants (reduce or eliminate unpleasant odor and protect against the formation of malodor on body surfaces), emollients (help to maintain the soft, smooth, and pliable appearance of the skin by their ability to remain on the skin surface or in the stratum corneum to act as a lubricant, to reduce flaking, and to improve the skin's appearance), external analgesics (a topically applied drug that has a topical analgesic, anesthetic, or antipruritic effect by depressing cutaneous sensory receptors, or that has a topical counterirritant effect by stimulating cutaneous sensory receptors), film formers (to hold active ingredients on the skin by producing a continuous film on the skin upon drying), fragrances (consumer appeal), humectants (increase the water content of the top layers of the skin), natural moisturizing agents (NMA) and other skin moisturizing ingredients known in the art, opacifiers (reduce the clarity or transparent appearance of the product), skin conditioning agents, skin exfoliating agents (ingredients that increase the rate of skin cell turnover: alpha hydroxy acids and beta hydroxyacids), skin protectants (a drug product which protects injured or exposed skin or mucous membrane surface from harmful or annoying stimuli), solvents (liquids employed to dissolve components found useful in the cosmetics or drugs), sunscreens (ingredient that absorbs at least 85% of the light in the UV range at wavelengths from 290 to 320 nonometers, but transmit UV light at wavelengths longer than 320 nanometers), and surfactants (as cleansing agents, emulsifying agents, solubilizing agents, and suspending agents).

In addition to these classes of ingredients, small amounts (from about 0.01 to about 20%) of oil soluble/dispersible or lipophilic materials can be easily emulsified into the formulation using anionic, cationic, nonionic and/or zwitterionic surfactants. Lipophilic materials without limitation include oils (minerals, vegetable, and animal), fatty esters and the like. Powders to enhance lubricity, oil absorption, provide skin protection, astringency, opacity, etc. and microencapsulated ingredients can also be dispersed into the formulation.

In an alternative embodiment of the present invention, the beneficial chemical compounds used in the present invention may not require transfer to a consumer's skin in order to provide specific benefits to the consumer. In this embodiment, the beneficial chemistry agents can be attached to the amino functional siloxane rather than the non-amino functional siloxane. In fact, the non-amino functional siloxane may not be required in the siloxane treatment. As described above, the amino functional siloxane chemically bonds to the paper product. Thus, since the beneficial chemical compound is attached to the amino functional siloxane, no transfer of the chemical compound will occur.

In yet another alternative embodiment of the present invention, a combination of beneficial chemistry agents can be used in which some require transfer to a consumer's skin to impart specific benefits to the consumer and some which do not require transfer. In this embodiment, the beneficial chemistry agents that require transfer to a consumer's skin are bonded to the non-amino functional siloxane. The beneficial chemistry agents that do not require transfer to a consumer's skin, on the other hand, are bonded to the amino functional siloxane. Again, since siloxanes readily bond to most compounds, various different and diverse beneficial chemistry agents can be used and combined as desired.

In still a further alternative embodiment of the present invention, a modified non-amino functional siloxane is incorporated into a lotion applied to a facial tissue or other nonwoven material. Transfer of the beneficial chemical compound to the consumer is effected with the transfer of the lotion from the facial tissue to the consumer. In this embodiment, a modified non-amino functional siloxane is incorporated into a lotion. The siloxane tends to migrate to the surface by a process commonly known as hysteresis or the retardation of a chemical system from reaching equilibrium. In this migration to the surface, the modified siloxane moves to the air/substrate interface where the beneficial chemical compounds will most easily be transferrable to the consumer's skin.

Any suitable lotion may be used with the siloxane treatment of the present invention. Particular examples are disclosed in U.S. Pat. Nos. 5,601,871; 5,614,293; 5,665,426; 5,885,697; 5,650,218; and 5,869,075 which are all incorporated herein by reference. The lotion can be water-based or oil-based. Suitable water based compositions include, but are not limited to, emulsions and water-dispersible compositions which can contain, for example, debonders (cationic, anionic or nonionic surfactants), or polyhdroxy compounds such as glycerin or propylene glycol. The basesheet could be treated with a bi-component system comprising a debonder and a polyhydroxy compound. Both components can be added separately or mixed together prior to being applied to the basesheet.

Oil-based compositions can include combinations of oil and wax. In particular embodiments, the tissue products are made by applying, on the surface(s) of the tissue, large numbers of individual deposits of a melted moisturizing/protective additive composition comprising a wax and an oil, and thereafter resolidifying the composition to form a distribution, of solid deposits on the surface(s) of the tissue. Because the composition is a solid at room temperature and rapidly solidifies after deposition, it has less tendency to penetrate and migrate into the sheet. Compared to tissues treated with liquid formulations, this leaves a greater percentage of the lotion on the surface of the tissue where it can contact and/or transfer to the user's skin to provide a benefit. Thus, a lower add-on amount can be used to deliver the same benefit at lower cost because of the efficient placement of the composition substantially at the surface of the product.

The lotion may comprise solidified deposits of a composition comprising from about 30 to about 90 weight percent oil, and from about 10 to about 40 weight percent wax, preferably also containing from about 5 to about 40 weight percent fatty alcohol. The composition can have a melting point of from about 30° C. to about 70° C., more specifically from about 40° C. to about 60° C. For purposes herein, “melting point” is the temperature at which the majority of the melting occurs, it being recognized that melting actually occurs over a range of temperatures.

The amount of oil in the composition can be from about 30 to about 90 weight percent, more specifically from about 40 to about 70 weight percent, and still more specifically from about 45 to about 60 weight percent. Suitable oils include, but are not limited to, the following classes of oils: petroleum or mineral oils, such as mineral oil and petrolatum; animal oils, such as mink oil and lanolin oil; plant oils, such as aloe extract, sunflower oil and avocado oil; and silicone oils which can be directly bonded to the beneficial chemistry agents, such as dimethicone and alkyl methyl silicones.

The amount of wax in the composition can be from about 10 to about 40 weight percent, more specifically from about 10 to about 30 weight percent, and still more specifically from about 15 to about 25 weight percent. Suitable waxes include, but are not limited to the following classes: natural waxes, such as beeswax and carnauba wax; petroleum waxes, such as paraffin and ceresine wax; silicone waxes, such as alkyl methyl siloxanes; or synthetic waxes, such as synthetic beeswax and synthetic sperm wax.

The amount of fatty alcohol in the composition, if present, can be from about 5 to about 40 weight percent, and more specifically from about 10 to about 30 weight percent. Suitable fatty alcohols include alcohols having a carbon chain length of C¹⁴-C³⁰, including acetyl alcohol, stearyl alcohol, behenyl alcohol, and dodecyl alcohol.

The total tissue add-on amount of the lotion can be from about 0.5 to about 40 weight percent, more specifically from about 3 to about 15 weight percent, and still more specifically from about 5 to about 10 weight percent, based on the weight of the tissue.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore those of ordinary skill in the art will appreciate that the foregoing description is by way of example only, and is not intended to limit the invention so further described in such appended claims.

What is claimed:

1. A paper product capable of transferring beneficial chemical compounds to an opposing surface comprising:
a nonwoven web containing pulp fibers, said nonwoven web having a first surface and a second and opposing surface; and
a siloxane treatment applied to at least one surface of said nonwoven web, said siloxane treatment comprising an amino functional siloxane composition bonded to said pulp fibers contained in said web, and a complex of a non-amino functional siloxane and at least one beneficial chemistry agent, the beneficial chemistry agent comprising a skin wellness composition selected from the group consisting of skin medicinal agents, skin therapeutic agents and antimicrobial agents, said non-amino functional siloxane and beneficial chemistry agent complex having an attraction to said amino functional siloxane composition, wherein said complex is configured to be transferred to an opposing surface when said opposing surface is contacted with said paper product.

2. A paper product as defined in claim 1, wherein said amino functional siloxane composition comprises an amine-modified polysiloxane having the following formula: 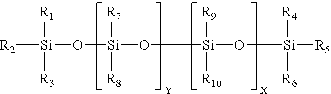
wherein X and Y are integers greater than 0 and the mole ratio of X to (X+Y) is from about 0.005 percent to about to about 25 percent, R₁, R₃, R₄, R₆-R₉ are alkyl groups, R₂ and R₅ are alkyl groups, hydroxyl groups or alkyl alcohol groups, and R₁₀ comprises an amine group, an imine group or an amide group.

3. A paper product as defined in claim 2, wherein said amino functional siloxane composition further comprises another modified polysiloxane having the following formula: 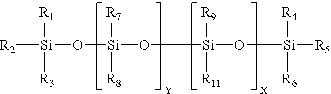
wherein X and Y are integers greater than 0 and the mole ratio of X to (X+Y) is from about 0.005 percent to about to about 25 percent, R₁, R₃, R₄, R₆-R₉ are alkyl groups, R₂ and R₅ are alkyl groups, hydroxyl groups or alkyl alcohol groups, and R₁₁ comprises an ether, a polyether, an ester, an amine, an imine, an amide, or alkyl and alkenyl analogues thereof.

12. A paper product as defined in claim 2, wherein said amino-modified polysiloxane has a viscosity of from about 25 centipoise to about 200,000 centipoise.

5. A paper product as defined in claim 1, wherein said non-amino functional siloxane comprises a material selected from the group consisting of an alkyl siloxane, a hydroxyl siloxane, and a hydrogen saturated siloxane.

6. A paper product as defined in claim 1, wherein said siloxane treatment comprises an emulsion.

7. A paper product as defined in claim 1, wherein said siloxane treatment is printed on to said surface of said nonwoven web.

8. A paper product as defined in claim 1, further comprising a second beneficial chemistry agent that is bonded to said amino functional siloxane composition.

9. A paper product as defined in claim 1, wherein said siloxane treatment is applied to said nonwoven web in an amount from about 0.1 percent to about 50 percent by weight.

10. A paper product as defined in claim 1, wherein said siloxane treatment is applied to said nonwoven web in an amount from about 0.1 percent to about 5.0 percent by weight.

11. A paper product as defined in claim 1, wherein said beneficial chemistry agent comprises a material selected from the group consisting of an anti-inflammatory compound, a lipid, an inorganic anion, an inorganic cation, a protease inhibitor, a sequestration agent, and mixtures thereof.

12. A paper product as defined in claim 1, wherein the amino functional siloxane composition is generally hydrophobic.

13. A facial tissue capable of transferring beneficial chemical compounds to the skin of a user comprising:
a nonwoven web containing pulp fibers, said nonwoven web having a first surface and a second and opposing surface, said nonwoven web having a basis weight of from about 4 to about 40 pounds per ream;
a siloxane treatment applied to at least one surface of said nonwoven web, said siloxane treatment comprising an amino functional siloxane composition bonded to said pulp fibers contained in said web, and a complex of a non-amino functional siloxane and a beneficial chemistry agent, said non-amino functional siloxane and beneficial chemistry agent complex having an attraction to said amino functional siloxane composition, the beneficial chemistry agent comprising a skin wellness composition selected from the group consisting of skin medicinal agents, skin therapeutic agents and antimicrobial agents, wherein said siloxane and beneficial chemistry agent complex is configured to be transferred to the skin of a user when the facial tissue is contacted with the skin.

14. A facial tissue as defined in claim 13, wherein said non-amino functional siloxane comprises a material selected from the group consisting of an alkyl siloxane, a hydroxyl siloxane, and a hydrogen saturated siloxane.

15. A facial tissue as defined in claim 13, wherein said non-amino functional siloxane comprises a methyl dimethyl siloxane.

16. A facial tissue as defined in claim 13, wherein said non-amino functional siloxane comprises a polydimethyl siloxane.

17. A facial tissue as defined in claim 13, wherein said facial tissue comprises a plurality of plies, one of said plies comprising said nonwoven web.

18. A facial tissue as defined in claim 13, wherein said siloxane treatment is printed on to said surface of said nonwoven web.

19. A facial tissue as defined in claim 13, wherein said siloxane treatment is applied to said nonwoven web in an amount from about 0.1 percent to about 50 percent by weight.

20. A facial tissue as defined in claim 13, wherein said siloxane treatment is applied to said nonwoven web in an amount from about 0.1 percent to about 5.0 percent by weight.

21. A facial tissue as defined in claim 13, wherein said beneficial chemistry agent comprises a material selected from the group consisting of an anti-inflammatory compound, a lipid, an inorganic anion, an inorganic cation, a protease inhibitor, a sequestration agent, and mixtures thereof.

22. A facial tissue as defined in claim 13, wherein the amino functional siloxane composition is generally hydrophobic.
